(19) United States
(12) Patent Application Publication
(10) Pub. No.: US 2023/0023987 A1
(43) Pub. Date: Jan. 26, 2023

(54) METHODS FOR TREATING CANCER COMPRISING LOW DOSE RADIATION

(71) Applicant: Board of Regents, The University of Texas System, Austin, TX (US)

(72) Inventors: James W. WELSH, Houston, TX (US); Hampartsoum B. BARSOUMIAN, Houston, TX (US); Maria Angelica CORTEZ, Houston, TX (US)

(73) Assignee: Board of Regents, The University of Texas System, Austin, TX (US)

(21) Appl. No.: 17/757,730

(22) PCT Filed: Dec. 18, 2020
(86) PCT No.: PCT/US2020/066200

§ 371 Date: Jun. 20, 2022

Related U.S. Application Data
(60) Provisional application No. 62/951,964, filed on Dec. 20, 2019.

(51) Int. Cl. A61N 5/10; A61P 35/00; C07K 16/28; A61K 31/7076; A61K 35/17; A61K 31/675; A61K 39/395; A61P 11/00

(52) U.S. Cl. CPC A61N 5/1077; A61P 35/00; C07K 16/2818; A61K 31/7076; A61K 35/17; A61K 31/675; A61K 39/3955; A61P 11/00; A61K 2039/505

(57) ABSTRACT

The present disclosure provides methods of treating cancer in a patient comprising administering a combination of a low dose radiotherapy and an immune checkpoint inhibitor therapy. The patient may be further administered a cell therapy, such as chimeric antigen receptor T-cell therapy or chimeric antigen receptor NK-cell therapy. The low dose radiation modulates the tumor microenvironment of solid tumors to allow better efficacy, activation, and infiltration of the anti-tumor effector immune cells.

This application claims benefit of priority to U.S. Provisional Application Ser. No. 62/951,964, filed Dec. 20, 2019, the entire contents of which are hereby incorporated by reference.

### BACKGROUND

### 1. Field

The present invention relates generally to the field of molecular biology and medicine. More particularly, it concerns methods of treating cancer with low dose radiation.

### 2. Description of Related Art

The first checkpoint inhibitor approved for NSCLC, an antibody to the programmed death 1 (PD1) receptor, inhibits a membrane protein expressed on T cells, B cells, NK cells, activated monocytes, and DCs. The second common checkpoint inhibitor, anti-cytotoxic T-lymphocyte associated protein 4 (CTLA-4), is targeted to a receptor expressed on T cells, extensively so on Tregs. When CTLA-4 is bound by CD80 or CD86 on an antigen-presenting cell, it transmits an inhibitory signal to T cells but promotes Treg immunosuppressive functions. Because TILs consist of CD4+ and CD8+ lymphocytes, some TILs directly destroy tumors and others promote stimulation of other immune cells (B cells, macrophages, CD8+ T cells) to promote tumor-cell lysis. However, despite current advances with immunotherapy, the majority of solid tumors remain refractory to treatment due to the inhibitory nature of their stroma that mediates immune evasion. Thus, there is an unmet need for improved immunotherapies for the treatment of cancer.

### SUMMARY

In certain embodiments, the present disclosure a provide method for treating cancer in a subject comprising administering low dose radiotherapy in combination with an effective amount of an immune checkpoint inhibitor to the subject. In another embodiment, there is provided a combination of low dose radiotherapy with an effective amount of an immune checkpoint inhibitor for use in a method for the treatment of cancer in a subject. In particular aspects, the method does not comprise administering a high dose radiotherapy.

In some aspects, the low dose radiation is an external-beam radiation therapy (XRT), proton beam therapy or brachytherapy. In certain aspects, the immune checkpoint inhibitor is administered to the subject after the low dose radiotherapy. In some aspects, the low dose radiation modulates the tumor microenvironment for increased anti-tumor M1 macrophage polarization, increased tumor infiltrating lymphocytes, increased NK cells, and/or decreased TGFβ. In particular aspects, the low dose radiation results in increased CXCL1, CXCL16, CXCL12, CXCR2P1, CCR1, CCR7, IL6R, IL1RAP, IL17RC, IL17RA, IL4R, IL13RA1, IL33, ILiR1, IL12RB1, IL10RB, IL10RA, and/or IL21R. In specific aspects, the low dose radiation is administered at a dosage of 20-160 cGy per fraction per day. In some aspects, the low dose radiation is administered at a final dose of about 0.1-12 Gy (e.g., 0.2, 0.5, 1., 2, 3, 4, 5, 6, 7, 8, 9, 10, or 22 Gy). In some aspects, the low dose radiation is administered at a final dose of about 1-9 Gy (e.g., 2, 3, 4, 5, 6, 7, or 8 Gy). In particular aspects, the low dose radiation is administered at a dosage of 1.4 Gy for 5 days.

In certain aspects, the at least one checkpoint inhibitor is selected from an inhibitor of CTLA-4, PD-1, PD-L1, PD-L2, LAG3, BTLA, B7H3, B7H4, TIM3, KIR, or A2aR. In particular aspects, the immune checkpoint inhibitor is anti-PD1 therapy, anti-PDL1 therapy, anti-CTLA-4, anti-TIGIT therapy, anti-LAG3 therapy, anti-TIM3 therapy, or anti-GITR therapy. In specific aspects, the at least one checkpoint inhibitor is an anti-PD-1 antibody, anti-PD-L1 antibody, anti-PD-L2 antibody, anti-CTLA-4 antibody, and/or anti-KIR antibody. For example, the immune checkpoint inhibitor is nivolumab, pembrolizumab, pidilizumab, tremelimumab, ipilimumab, lirilumab AMP-514, REGN2810, CT-011, BMS 936559, MPDL3280A AMP-224, durvalumab, atezolizumab, alemtuzumab, avelumab, rHIgM12B7, IMP321, BMS-986016, or PBF-509.

In additional aspects, the method comprises administering more than one immune checkpoint inhibitor. In some aspects, the subject has been previously administered an immunotherapy, such as 1-5 days before, 1-3 weeks before or 1-6 months before the present therapy. In some aspects, the immunotherapy is cell therapy or immune checkpoint inhibitor therapy. In specific aspects, the subject had low or no response to the immunotherapy.

In further aspects, the method further comprises administering a cell therapy. In some aspects, the cell therapy comprises T cells, NK cells, or dendritic cells. In particular aspects, the cell therapy comprises cells engineered to express a T cell receptor (TCR) or chimeric antigen receptor (CAR).

In some aspects, the method further comprises an additional anti-cancer therapy. In certain aspects, the additional anti-cancer therapy comprises chemotherapy, radiotherapy, gene therapy, surgery, hormonal therapy, anti-angiogenic therapy or immunotherapy. In particular aspects, the additional anti-cancer therapy comprises chemotherapy. For example, the chemotherapy is fludarabine or cyclophosphamide.

In certain aspects, the immunotherapy comprises an OX40 agonist, an IDO inhibitor, an anti-MERTK immunotherapy, an intratumoral injection, a STING-targeted immunotherapy, a NLRP3-targeted immunotherapy, a TLR9-targeted immunotherapy, a CPG-targeted immunotherapy, a TLR4-targeted immunotherapy, a TLR7/8-targeted immunotherapy, a OX40-targeted immunotherapy, a 4-1BB-targeted immunotherapy, a MER-TK-targeted immunotherapy, an oncolytic virus immunotherapy, an anti-CD40 immunotherapy, a FLT-3-ligand immunotherapy, or cytokine immunotherapies, such as IL-2, IL-12, and/or IL-15.

In some aspects, the subject is a human. In certain aspects, the cancer is a lung cancer, a brain cancer, a breast cancer, a head and neck cancer, a cervical cancer, prostate cancer, a cancer of the eye, or a thyroid cancer. In some aspects, the low dose radiation and/or immune checkpoint inhibitor are administered two or more times.

A further embodiment provides a method for modulating the tumor microenvironment to increase cytokines and chemokines that favor infiltration of effector immune cells comprising administering a low dose radiation therapy. In some aspects, the low dose radiation modulates the tumor microenvironment for increased anti-tumor M1 macrophage polarization, increased tumor infiltrating lymphocytes, increased NK cells, and/or decreased TGFβ. In certain aspects, the low dose radiation results in increased CXCL1, CXCL16, CXCL12, CXCR2P1, CCR1, CCR7, IL6R, IL1RAP, IL17RC, IL17RA, IL4R, IL13RA1, IL33, IL1R1, IL12RB1, IL10RB, IL10RA, and/or IL21R.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Percentage of NK cells in tumor infiltrating lymphocytes in response to radiation. Low-dose radiation improves NK-cell percentages in TILs. Spl=spleen; dLN=draining lymph node; TIL=tumor infiltrating leukocytes.

FIG. 2: Percentage of M1 macrophages in Gr1+CD11b+ tumor infiltrating lymphocytes. Low-dose radiation favors the polarization of anti-tumor M1 macrophages in the tumor microenvironment.

FIG. 3: Level of TGFβ1 expression in response to radiation. Low-dose radiation downregulates TGF-β cytokine level in tumors treated with L-XRT vs. untreated controls.

FIG. 4: Percentage survival of mice treated with low dose radiation alone or in combination with immune checkpoint therapy. In 344SQ murine lung adenocarcinoma model, low-dose radiation significantly improves anti-tumor outcomes of checkpoint inhibitors such as anti-PD1 and anti-CTLA-4.

FIG. 5: Percentage survival of mice treated with radiation alone or in combination with chemotherapy. Preconditioning of 129Sv/Ev tumor-bearing mice with 2 cycles of low dose cyclophosphamide (60 mg/kg) followed by 4 cycles of low dose Fludarabine (50 mg/kg) significantly improved the survival of mice subsequently treated with low-dose radiation.

### DESCRIPTION OF ILLUSTRATIVE EMBODIMENTS

Despite current advances with immunotherapy, the majority of solid tumors remain refractory to treatment due to the inhibitory nature of their stroma that mediates immune evasion. The stroma is rich with inhibitory cell populations such as regulatory T cells (Tregs), myeloid derived suppressor cells (MDSCs), pro-tumor growth M2 macrophages, and cancer associated fibroblasts (CAFs). The present studies showed that low-dose radiation (L-XRT) is capable of modulating the tumor microenvironment (TME) to favor anti-tumor M1 macrophage polarization and downregulation of the inhibitory cytokine TGF-β. L-XRT also increased the percentages of natural killer (NK) cells and effector T-cells in the TME leading to retardation of tumor growth.

Moreover, L-XRT modulated the chemokine signature in both preclinical and clinical cases tested to facilitate the infiltration of effector immune cells. For the preclinical model, 129Sv/Ev mice were injected subcutaneously with 344SQ lung adenocarcinoma cells. Tumors were subjected to local L-XRT (2Gy total in 1Gy fractions) when they reached around 8 mm in diameter. Tumors were harvested 24 hours after the last L-XRT fraction and RNA from tumor infiltrating leukocytes (TILs) was used to run immuno-NanoString panel to assess the cytokines and chemokines produced. Amongst the significantly upregulated targets were CXCL13, CCL8, CCL11, CXCL12, IL33, and IL1r1. For the clinical cases, patient biopsies were collected at baseline and after L-XRT (approximately 1.4Gy×5 days for a total of 7 Gy), total RNA was isolated and subjected to RNA sequencing analysis. Key upregulated ligands and receptors post L-XRT included CXCL1, CXCL16, CXCL12, CXCR2P1, CCR1, CCR7, TL6R, IL1RAP, IL17RC, IL17RA, TL4R, IL13RA1, IL33, IL1R1, IL12RB1, IL10RB, IL10RA, and IL21R. Most of these factors are involved in the chemoattraction and potential activation of T cells, B cells, and myelocytes, such as monocytes, to better infiltrate and overcome tumor immune evasion. It was found that low dose radiation can overcome the tumor inhibitory stroma and generate soluble factors that may be utilized to design novel cell therapies. Moreover, activated immune cells expressing higher levels of chemokine ligands or their corresponding receptors can be attracted specifically to the tumor site rather than being dispersed to secondary organs lacking tumor lesions.

Accordingly, the present disclosure provides methods for the use of low dose radiation to modulate the tumor microenvironment and generate cytokines or cytokine receptors and chemokines or chemokine receptors that favor the infiltration of effector immune cells to kill tumors. Low dose radiation can act on tumor and its associated environment to generate pulling forces of effector immune cells through the selective production of chemokines and cytokines. The low dose radiation can diversify and intensify the chemokines produced to extend the therapeutic benefit to solid tumors with different histologies and activate T cells and NK cells. Low dose radiotherapy can be administered in combination with engineered cell therapies, such as CAR-T cells, CAR-NK cells, or TCR engineered cells, to enhance their penetration into solid tumors, hence maximizing the efficacy and persistence of adoptively transferred cellular therapies. Low dose radiation may be administered in combination with checkpoint inhibitors, such as anti-PD1, anti-PDL1, anti-TIGIT, anti-GITR, anti-CTLA-4, anti-LAG3, anti-TIM3, or anti-Siglec-15 to enhance T cell activation and infiltration to tumors. In some embodiments, the present methods are used to treat patients with solid tumors that have been previously administered a cell therapy or immune checkpoint inhibitor therapy and had low or no response to the therapy.

### I. Definitions

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.”

The term “essentially” is to be understood that methods or compositions include only the specified steps or materials and those that do not materially affect the basic and novel characteristics of those methods and compositions.

The term “substantially free of” is used to 98% of the listed components and less than 2% of the components to which composition or particle is substantially free of.

The terms “substantially” or “approximately” as used herein may be applied to modify any quantitative comparison, value, measurement, or other representation that could permissibly vary without resulting in a change in the basic function to which it is related.

The term “about” means, in general, within a standard deviation of the stated value as determined using a standard analytical technique for measuring the stated value. The terms can also be used by referring to plus or minus 5% of the stated value.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

### II. Methods of Treatment

Further provided herein are methods for treating or delaying progression of cancer in an individual comprising administering low dose radiation to the subject in combination with an immunotherapy, such as an immune checkpoint inhibitor.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is non-small cell lung cancer.

In some embodiments, a radiation therapy as disclosed herein is administered to treat a primary cancer. In some embodiments, while the radiation therapy may not be part of the main treatment for a cancer type, it may nonetheless be used to treat tumors that have spread to other parts of the body (e.g., metastatic tumors that have spread to the brain, spinal fluid, or testicles, or lung, etc.). The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and/or hairy cell leukemia.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

A. Radiotherapy

Radiotherapy is the use of high-energy radiation from x-rays, gamma rays, neutrons, protons, and other sources to kill cancer cells and shrink tumors. Radiotherapy may also be called irradiation and radiation therapy. In some embodiments, a low dose radiotherapy is administered to a subject to modulate the tumor microenvironment.

In some embodiments, repeated administration of a low dose radiotherapy are administered to a subject to treat a cancer. X-rays, gamma rays, and charged particles are examples of types of radiation used for cancer treatment. The radiation may be delivered by a machine outside the body (external-beam radiation therapy (XRT)), or it may come from radioactive material placed in the body near cancer cells (internal radiation therapy, also called brachytherapy). In some embodiments, the radiotherapy is an XRT, and the radiotherapy may be administered to the subject at a low dosage (e.g., of about 0.2-1.9 Gy per fraction for a total dose of about 2-12Gy). The subject may receive a second anti-cancer therapy, such as an immunotherapy, in combination with the low dose XRT (e.g., on the same day or within 1, 2, or 3 days).

Radiotherapy includes external-beam radiation therapy; internal radiation therapy (brachytherapy), and systemic radiation therapy. Types of external-beam radiation therapy include: Intensity-modulated radiation therapy (IMRT), Image-guided radiation therapy (IGRT), tomotherapy, stereotactic radiosurgery, stereotactic body radiation therapy, proton therapy, and other charged particle beams. Systemic radiation therapy uses radioactive substances, such as radioactive iodine or a radiolabeled monoclonal antibody, that travel in the blood and/or to tissues throughout the body to kill cancer cells.

Radiation therapy is a primary therapy for treating patients with various cancers such as, e.g., inoperable localized non-small cell lung carcinoma. However, there is a high rate of local failure, and while it increases median survival, the therapy is often not curative. Standard radiation fractionation provides a daily dose on the order of 1.8-2Gy, to a final dose of 60-70Gy. By contrast, a Stereotactic Body Radiation Therapy (SBRT), also referred to as a stereotactic ablative radiotherapy (SABR), is a relatively novel technique in radiation therapy of lung carcinomas, delivering the total dose in 5 or fewer treatments of radiation (hypofractionation). Response rates in clinical trials suggest SBRT could be an important therapeutic advance. This approach may have significant relevance to the endogenous immune response, since lymphocytes are sensitive to even low radiation doses and are cleared rapidly from the radiation field. Standard fractionated radiation treatment may limit the effectiveness of the immune system by constantly removing tumor antigen-specific T cells at the target site. Thus, although standard fractionation has been shown to generate endogenous anti-tumor immune responses, SBRT hypofractionation may in some embodiments provide benefits when combined with an immunotherapy. In traditional external beam radiation therapy coupled with radiosensitizer administration, a beam of high energy X-rays, generated outside the patient by a linear accelerator, is delivered to a tumor. Most body tissue does not absorb or block X-rays, so they progress through the body, constantly releasing energy. When the cancer tumor is within the path of the X-ray, it receives some of that radiation; however, surrounding healthy tissue receives radiation as well. In order to limit the extent of collateral tissue damage, oncologists typically bombard the tumor area with the lowest level of effective radiation from many different points of entrance in an attempt to minimize damage to normal tissues. Even modem external beam radiation systems with improved real-time imaging of the patient anatomy will inevitably treat substantial normal tissue volumes when targeting the tumor.

Other energy sources, such as particle beams contain charged atomic particles. Particle beams have tremendous energy but also high mass and as such they slow down as they encounter body tissue. Particles can be controlled, for example, to release their energy at a specific point in the body. Particle beam therapy uses electrons, neutrons, heavy ions (such as protons, carbon ions and helium); and pi-mesons (also called pions).

Various radiotherapy methods that utilize γ-rays, X-rays may be used. In some instances, it is anticipated that as microwaves, proton beam irradiation (U.S. Pat. Nos. 5,760,395 and 4,870,287), or UV-irradiation may be used as a radiotherapy. In some embodiments, directed delivery of radioisotopes to tumor cells may also be used.

As used herein, a “high dose” or “higher dose” radiotherapy or radiation therapy, such as an XRT, refers to a cumulative external irradiation of a patient in a dose of about of about 10-90 Gy, more preferably about 10-60, 10-50, 10-45, 10-40, 15-40, 15-35, 25-35, 30-40, 10-35, 10-25, 10-20, 15-30 Gy, or about 10, 15, 20, 25, 30, 35, 40 Gy, or any range derivable therein. The higher dose radiotherapy may provide about 1.8-2Gy per fraction.

As used herein, a “low dose” or “lower dose” radiotherapy or radiation therapy, such as an XRT, refers to a dosage of 0.1-1.9 Gy per fraction, or about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9 Gy per fraction, or any range derivable therein. The low dose radiotherapy may comprise a total dose of about 1-13, 2-12, 1-9, 2-9, 2-10, 1, 2, 3, 4, 5, 6, 7, 8, 9, 10, 11, or 12 Gy, or any range derivable therein. The low dose radiotherapy may be separated into separate administrations (e.g., 1, 2, 3, 4, or 5 administrations of the radiotherapy over a period of about 1, 2, 3, 4, or 5 days). The low dose radiotherapy may provide a dose of 10-170, 10-160, 20-160, 30-140, or 10, 20, 30, 40, 50, 60, 70, 80, 90, 100, 110, 120, 130, 140, 150, 160, or 170 cGy per fraction, or any range derivable therein. In some preferred embodiments, the low dose radiotherapy is a sub-clinical dose of XRT (e.g., 20-190 cGy per fraction, for a total of 2-12 Gy). The low dose radiotherapy or XRT may be delivered, e.g., in 1, 2, 3, 4, or 5 fractions. The lower dose radiotherapy may be a SBRT.

Radiation therapy may be stereotactic body radiotherapy, or SBRT. Stereotactic radiotherapy uses essentially the same approach as stereotactic radiosurgery to deliver radiation to the target tissue; however, stereotactic radiotherapy generally uses multiple small fractions of radiation as opposed to one large dose, but certain applications of SBRT may still be accomplished with a single fraction. Stereotactic body radiotherapy may be used to treat tumors, e.g., in the brain, lung, liver, pancreas, prostate, spine, as well as other parts of the body.

Radiotherapy may be used for curative, adjuvant, or palliative treatment. Suitable types of radiotherapy include conventional external beam radiotherapy, stereotactic radiation therapy (e.g., Axesse, Cyberknife, Gamma Knife, Novalis, Primatom, Synergy, X-Knife, TomoTherapy or Trilogy), Intensity-Modulated Radiation Therapy, particle therapy (e.g., proton therapy), brachytherapy, delivery of radioisotopes, intraoperative radiotherapy, Auger therapy, Volumetric modulated arc therapy (VMAT), Virtual simulation, 3-dimensional conformal radiation therapy, and intensity-modulated radiation therapy, etc.

The radiotherapy may be administered in combination with a second anti-cancer therapy, such as an immunotherapy, e.g., an anti-CTLA-4 compound or antibody, an anti-OX40 antibody, an anti-4-1BB antibody, or an anti-PD-1 antibody. Anti-OX40 and anti-4-1BB antibodies include agonist antibodies, and anti-PD-1 antibodies include antagonist antibodies, e.g., as described in WO2018150326. In some embodiments, the radiation therapy used is a SBRT. The cancer may be, e.g., an anti-PD-1 resistant cancer. Suitable examples of radiation therapy include, for example, external beam radiotherapy (EBRT or XRT) or teletherapy, brachytherapy or sealed source radiotherapy, or systemic radioisotope therapy or unsealed source radiotherapy. In some embodiments, an anti-PD-1 ABP (e.g., an antagonist antibody) is included in the combination.

B. Immune Checkpoint Therapy

In some embodiments, the present methods comprise the combination of a low dose radiotherapy and an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129; International Patent Publication Nos. WO 01/14424, WO 98/42752, and WO 00/37504 (CP675,206, also known as tremelimumab; formerly ticilimumab); and U.S. Pat. No. 6,207,156 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application Nos. WO2001014424, and WO2000037504, and U.S. Pat. No. 8,017,114; all incorporated herein by reference.

An exemplary anti-CTLA-4 antibody is ipilimumab (also known as 10DI, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

C. Combination Therapies

In certain embodiments, the compositions and methods of the present embodiments involve low dose radiation in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The low dose radiation therapy may be administered before, during, after, or in various combinations relative to an additional cancer therapy, such as immune checkpoint therapy. The administrations may be in intervals ranging from concurrently to minutes to days to weeks. In embodiments where the low dose radiotherapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below low dose radiotherapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

2. Immunotherapy

In some preferred embodiments, low dose radiotherapy as described herein is administered in combination with an immunotherapy. Various immunotherapies are known that may be used in combination with a low dose radiotherapy as disclosed herein, including, e.g., anti-PD1 antibodies or compounds, anti-PD-L1 antibodies or compounds, anti-CTLA-4 antibodies or compounds, OX40 agonists, 4-1BB agonists, IDO inhibitors, Arginase inhibitors, anti-GITR antibodies or compounds, anti-LAG3 antibodies or compounds, anti-TIM3 antibodies or compounds, anti-TIGIT antibodies or compounds, and anti-MERTK antibodies or compounds, an oncolytic virus immunotherapy, intratumoral injections; immunotherapies targeting STING, NLRP3, TLR9, CPG, TLR4, TLR7/8, OX40, 4-1BB, or MER-TK; an anti-CTLA-4, anti-PD1, anti-PDL1, or anti-CD40 immunotherapy; FLT-3-ligand immunotherapies, and/or cytokine immunotherapies including IL-2, IL-12, and IL-15. Additionally, the low dose radiotherapy could be combined with cell therapies, such as T cells, NK cells, or dendritic cells that may be engineered to express a CAR or TCR.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, gp100, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311).

In some embodiments, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy. In one aspect, the adoptive T cell therapy comprises autologous and/or allogenic T cells. In another aspect, the autologous and/or allogenic T cells are targeted against tumor antigens.

3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

4. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Characterization of Low Dose Radiotherapy

L-XRT modulated the chemokine signature in both preclinical and clinical cases tested to facilitate the infiltration of effector immune cells. For the preclinical model, 129Sv/Ev mice were injected subcutaneously with 344SQ lung adenocarcinoma cells. Tumors were subjected to local L-XRT (2Gy total in 1Gy fractions) when they reached around 8 mm in diameter. Tumors were harvested 24 hours after the last L-XRT fraction and RNA from tumor infiltrating leukocytes (TILs) was used to run immuno-NanoString panel to assess the cytokines and chemokines produced. Amongst the significantly upregulated targets were CXCL13, CCL8, CCL11, CXCL12, IL33, and IL1r1 (Table 1).

TABLE 1
Molecular targets modulated in
murine post low-dose radiation

| Gene | Regulation status |
|---|---|
| CXCL13 | Up |
| CCL8 | Up |
| CCL11 | Up |
| CXCL12 | Up |
| IL33 | Up |
| IL1r1 | Up |
| IL6st | Up |
| IL1a | Down |
| IFNa2 | Down |
| CXCR4 | Down |
| CXCR2 | Down |
| CCRL2 | Down |
| IL1rn | Down |
| IL1r2 | Down |
| TGFb1 | Down |
| IL16 | Down |
| IFNa1 | Down |
| CCR10 | Down |
| IFNb1 | Down |
| IL25 | Down |

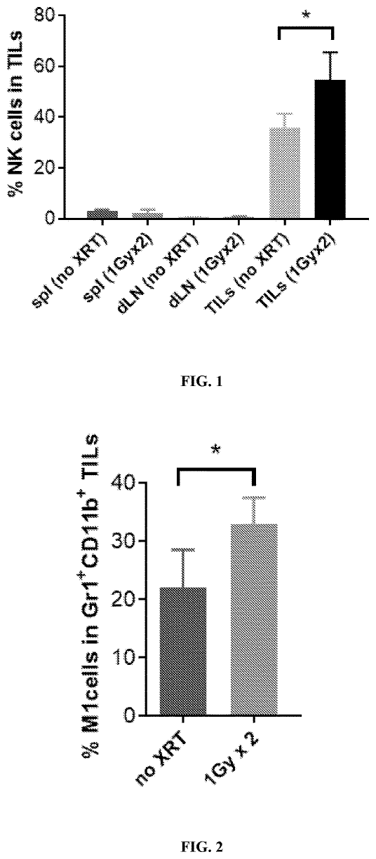

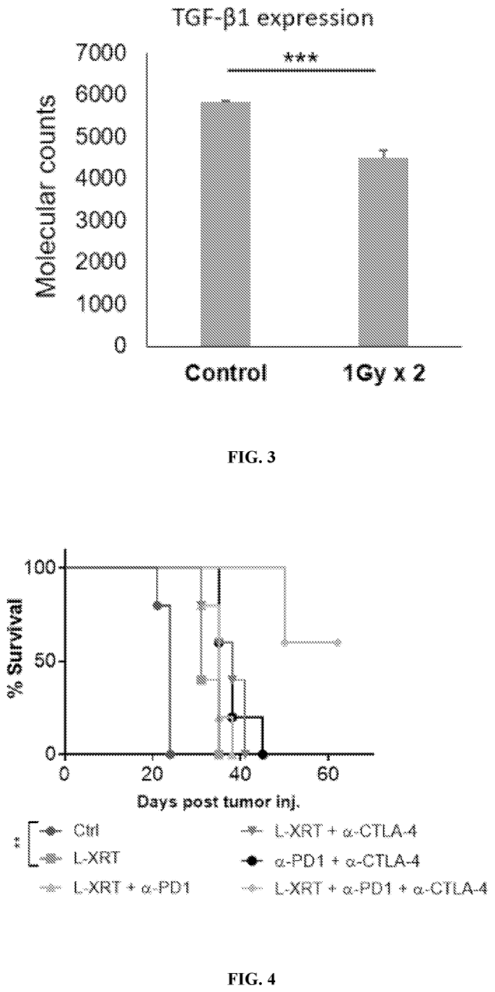

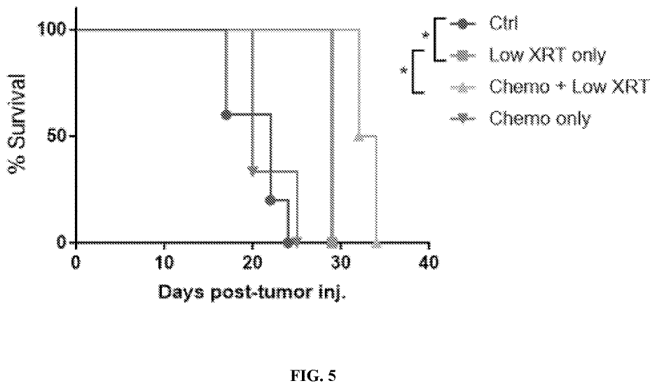

It was shown that the low-dose radiation improved NK-cell percentages in TILs (FIG. 1). In addition, it was observed that low-dose radiation favors the polarization of anti-tumor M1 macrophages in the tumor microenvironment (FIG. 2) and downregulates TGF-β cytokine level in tumors treated with L-XRT vs. untreated controls (FIG. 3). In 344SQ murine lung adenocarcinoma model, low-dose radiation significantly improved anti-tumor outcomes of checkpoint inhibitors such as anti-PD1 and anti-CTLA-4 (FIG. 4). Finally, preconditioning of 129Sv/Ev tumor-bearing mice with 2 cycles of low dose cyclophosphamide (60 mg/kg) followed by 4 cycles of low dose Fludarabine (50 mg/kg) significantly improved the survival of mice subsequently treated with low-dose radiation (FIG. 5).

For the clinical cases, patient biopsies were collected at baseline and after L-XRT (approximately 1.4Gy×5 days for a total of 7 Gy), total RNA was isolated and subjected to RNA sequencing analysis. Key upregulated ligands and receptors post L-XRT included CXCL1, CXCL16, CXCL12, CXCR2P1, CCR1, CCR7, IL6R, IL1RAP, IL17RC, IL17RA, IL4R, IL13RA1, IL33, IL1R1, IL12RB1, IL10RB, IL10RA, and IL21R (Table 2). Most of these factors are involved in the chemoattraction and potential activation of T cells, B cells, and myelocytes, such as monocytes, to better infiltrate and overcome tumor immune evasion. It was found that low dose radiation can overcome the tumor inhibitory stroma and generate soluble factors that may be utilized to design novel cell therapies.

TABLE 2
Selected molecular targets modulated in human
tumors (n = 4) post low-dose radiation.

| Gene | Regulation status |
|---|---|
| CXCL1 | Up |
| CXCL16 | Up |
| CXCL12 | Up |
| CXCR2P1 | Up |
| CCR1 | Up |
| CCR7 | Up |
| IL6R | Up |
| IL1RAP | Up |
| IL17RC, IL17RA | Up |
| IL4R | Up |
| IL13RA1 | Up |
| IL33 | Up |
| IL1R1 | Up |
| IL12RB1 | Up |
| IL10RB, IL10RA | Up |
| IL21R | Up |
| IL6st | Up |
| CXCL13 | Down |
| CXCL10 | Down |
| CXCL9 | Down |
| CXCR4 | Down |
| CXCR6 | Down |
| CXCR5 | Down |
| CXCR3 | Down |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Bukowski et al., Clinical Cancer Res., 4(10):2337-2347, 1998.
Christodoulides et al., Microbiology, 144(Pt 11):3027-3037, 1998
Davidson et al., J. Immunother., 21(5):389-398, 1998.
Hanibuchi et al., Int. J. Cancer, 78(4):480-485, 1998.
Hellstrand et al., Acta Oncologica, 37(4):347-353, 1998.
Hui and Hashimoto, Infection Immun., 66(11):5329-5336, 1998.
International Patent Publication No. WO1995001994
International Patent Publication No. WO1998042752
International Patent Publication No. WO2000037504
International Patent Publication No. WO2001014424
International Patent Publication No. WO2006/121168
International Patent Publication No. WO2009/101611
International Patent Publication No. WO2009/114335
International Patent Publication No. WO2010/027827
International Patent Publication No. WO2011/066342
International Patent Publication No. WO2015016718
International Patent Publication No. WO2018150326
Pardoll, Nat Rev Cancer, 12(4): 252-64, 2012.
Qin et al., Proc. Natl. Acad. Sci. USA, 95(24):14411-14416, 1998.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 6,207,156
U.S. Pat. No. 8,008,449
U.S. Pat. No. 8,017,114
U.S. Pat. No. 8,119,129
U.S. Pat. No. 8,329,867
U.S. Pat. No. 8,354,509
U.S. Pat. No. 8,735,553
U.S. Patent Publication No. US20110008369
U.S. Patent Publication No. US2014022021
U.S. Patent Publication No. US20140294898

What is claimed is:

1. A method of treating cancer in a subject comprising administering low dose radiotherapy in combination with an effective amount of an immune checkpoint inhibitor to the subject.

2. The method of claim 1, wherein the method does not comprise administering a high dose radiotherapy.

3. The method of claim 1 or 2, wherein the low dose radiation is an external-beam radiation, proton beam therapy, or brachytherapy.

4. The method of claim 1 or 2, wherein the low dose radiation is an external-beam radiation therapy (XRT).

5. The method of any of claims 1-4, wherein the immune checkpoint inhibitor is administered to the subject after the low dose radiotherapy.

6. The method of any of claims 1-5, wherein the low dose radiation modulates the tumor microenvironment for increased anti-tumor M1 macrophage polarization, increased tumor infiltrating lymphocytes, increased NK cells, and/or decreased TGFβ.

7. The method of any of claim 1-6, wherein the low dose radiation results in increased CXCL1, CXCL16, CXCL12, CXCR2P1, CCR1, CCR7, TL6R, IL1RAP, IL17RC, IL17RA, IL4R, IL13RA1, TL33, IL1R1, IL12RB1, IL10RB, IL10RA, and/or IL21R.

8. The method of any of claims 1-7, wherein the low dose radiation is administered at a dosage of 20-160 cGy per fraction per day.

9. The method of any of claims 1-8, wherein the low dose radiation is administered at a final dose of about 0.1-12 Gy.

10. The method of any of claims 1-9, wherein the low dose radiation is administered at a final dose of about 1-9 Gy.

11. The method of any of claims 1-10, wherein the low dose radiation is administered at a dosage of 1.4 Gy for 5 days.

12. The method of any of claims 1-11, wherein the at least one checkpoint inhibitor is selected from an inhibitor of CTLA-4, PD-1, PD-L1, PD-L2, LAG3, BTLA, B7H3, B7H4, TIM3, KIR, or A2aR.

13. The method of any of claims 1-11, wherein the immune checkpoint inhibitor is anti-PD1 therapy, anti-PDL1 therapy, anti-CTLA-4, anti-TIGIT therapy, anti-LAG3 therapy, anti-TIM3 therapy, or anti-GITR therapy.

14. The method of any of claims 1-13, wherein the at least one checkpoint inhibitor is an anti-PD-1 antibody, anti-PD-L1 antibody, anti-PD-L2 antibody, anti-CTLA-4 antibody, and/or anti-KIR antibody.

15. The method of any of claims 1-14, wherein the immune checkpoint inhibitor is nivolumab, pembrolizumab, pidilizumab, tremelimumab, ipilimumab, lirilumab AMP-514, REGN2810, CT-011, BMS 936559, MPDL3280A AMP-224, durvalumab, atezolizumab, alemtuzumab, avelumab, rHIgM12B7, IMP321, BMS-986016, or PBF-509.

16. The method of any of claims 1-15, wherein the method comprises administering more than one immune checkpoint inhibitor.

17. The method of any of claims 1-16, wherein the subject has been previously administered an immunotherapy.

18. The method of claim 17, wherein the immunotherapy is cell therapy or immune checkpoint inhibitor therapy.

19. The method of claim 17 or 18, wherein the subject had low or no response to the immunotherapy.

20. The method of any of claims 1-19, wherein the method further comprises administering a cell therapy.

21. The method of claim 20, wherein the cell therapy comprises T cells, NK cells, or dendritic cells.

22. The method of claim 21, wherein the cell therapy comprises NK cells.

23. The method of any of claims 20-22, wherein the cell therapy comprises cells engineered to express a T cell receptor (TCR) or chimeric antigen receptor (CAR).

24. The method of any of claims 1-23, wherein the method further comprises an additional anti-cancer therapy.

25. The method of claim 24, wherein the additional anti-cancer therapy comprises chemotherapy, radiotherapy, gene therapy, surgery, hormonal therapy, anti-angiogenic therapy or immunotherapy.

26. The method of claim 25, wherein the additional anti-cancer therapy comprises chemotherapy.

27. The method of claim 26, wherein the chemotherapy is fludarabine or cyclophosphamide.

28. The method of claim 25, wherein the immunotherapy comprises an OX40 agonist, an IDO inhibitor, an anti-MERTK immunotherapy, an intratumoral injection, a STING-targeted immunotherapy, a NLRP3-targeted immunotherapy, a TLR9-targeted immunotherapy, a CPG-targeted immunotherapy, a TLR4-targeted immunotherapy, a TLR7/8-targeted immunotherapy, a OX40-targeted immunotherapy, a 4-1BB-targeted immunotherapy, a MER-TK-targeted immunotherapy, an oncolytic virus immunotherapy, an anti-CD40 immunotherapy, a FLT-3-ligand immunotherapy, or cytokine immunotherapies.

29. The method of claim 28, wherein the cytokine immunotherapies are IL-2, IL-12, and/or IL-15.

30. The method of any of claims 1-29, wherein the subject is a human.

31. The method of any of claims 1-30, wherein the cancer is a lung cancer, a brain cancer, a breast cancer, a head and neck cancer, a cervical cancer, prostate cancer, a cancer of the eye, or a thyroid cancer.

32. The method of claim 28, wherein the low dose radiation and/or immune checkpoint inhibitor are administered two or more times.

33. A method for modulating the tumor microenvironment to increase cytokines and chemokines that favor infiltration of effector immune cells comprising administering a low dose radiation therapy.

34. The method of claim 33, wherein the low dose radiation modulates the tumor microenvironment for increased anti-tumor M1 macrophage polarization, increased tumor infiltrating lymphocytes, increased NK cells, and/or decreased TGFβ.

35. The method of claim 33, wherein the low dose radiation results in increased CXCL1, CXCL16, CXCL12, CXCR2P1, CCR1, CCR7, IL6R, IL1RAP, IL17RC, IL17RA, TL4R, IL13RA1, TL33, IL1R1, IL12RB1, IL10RB, IL10RA, and/or IL21R.

36. A combination of low dose radiotherapy with an effective amount of an immune checkpoint inhibitor for use in a method for the treatment of cancer in a subject.

37. The combination of claim 36, wherein the method does not comprise administering a high dose radiotherapy.

38. The combination of claim 36 or 37, wherein the low dose radiation is an external-beam radiation, proton beam therapy, or brachytherapy.

39. The combination of claim 36 or 37, wherein the low dose radiation is an external-beam radiation therapy (XRT).

40. The combination of any of claims 36-39, wherein the immune checkpoint inhibitor is administered to the subject after the low dose radiotherapy.

41. The combination of any of claims 36-40, wherein the low dose radiation modulates the tumor microenvironment for increased anti-tumor M1 macrophage polarization, increased tumor infiltrating lymphocytes, increased NK cells, and/or decreased TGFβ.

42. The combination of any of claim 36-41, wherein the low dose radiation results in increased CXCL1, CXCL16, CXCL12, CXCR2P1, CCR1, CCR7, TL6R, IL1RAP, IL17RC, IL17RA, TL4R, IL13RA1, TL33, IL1R1, IL12RB1, IL10RB, IL10RA, and/or IL21R.

43. The combination of any of claims 36-42, wherein the low dose radiation is administered at a dosage of 20-160 cGy per fraction per day.

44. The combination of any of claims 36-43, wherein the low dose radiation is administered at a final dose of about 0.1-12 Gy.

45. The combination of any of claims 36-44, wherein the low dose radiation is administered at a final dose of about 1-9 Gy.

46. The combination of any of claims 36-45, wherein the low dose radiation is administered at a dosage of 1.4 Gy for 5 days.

47. The combination of any of claims 36-46, wherein the at least one checkpoint inhibitor is selected from an inhibitor of CTLA-4, PD-1, PD-L1, PD-L2, LAG3, BTLA, B7H3, B7H4, TIM3, KIR, or A2aR.

48. The combination of any of claims 36-46, wherein the immune checkpoint inhibitor is anti-PD1 therapy, anti-PDL1 therapy, anti-CTLA-4, anti-TIGIT therapy, anti-LAG3 therapy, anti-TIM3 therapy, or anti-GITR therapy.

49. The combination of any of claims 36-48, wherein the at least one checkpoint inhibitor is an anti-PD-1 antibody, anti-PD-L1 antibody, anti-PD-L2 antibody, anti-CTLA-4 antibody, and/or anti-KIR antibody.

50. The combination of any of claims 36-49, wherein the immune checkpoint inhibitor is nivolumab, pembrolizumab, pidilizumab, tremelimumab, ipilimumab, lirilumab AMP-514, REGN2810, CT-011, BMS 936559, MPDL3280A AMP-224, durvalumab, atezolizumab, alemtuzumab, avelumab, rHIgM12B7, IMP321, BMS-986016, or PBF-509.

51. The combination of any of claims 36-50, wherein the method comprises administering more than one immune checkpoint inhibitor.

52. The combination of any of claims 36-51, wherein the subject has been previously administered an immunotherapy.

53. The combination of claim 52, wherein the immunotherapy is cell therapy or immune checkpoint inhibitor therapy.

54. The combination of claim 52 or 53, wherein the subject had low or no response to the immunotherapy.

55. The combination of any of claims 36-54, wherein the method further comprises administering a cell therapy.

56. The combination of claim 55, wherein the cell therapy comprises T cells, NK cells, or dendritic cells.

57. The combination of claim 56, wherein the cell therapy comprises NK cells.

58. The combination of any of claims 55-57, wherein the cell therapy comprises cells engineered to express a T cell receptor (TCR) or chimeric antigen receptor (CAR).

59. The combination of any of claims 36-58, wherein the method further comprises an additional anti-cancer therapy.

60. The combination of claim 59, wherein the additional anti-cancer therapy comprises chemotherapy, radiotherapy, gene therapy, surgery, hormonal therapy, anti-angiogenic therapy or immunotherapy.

61. The combination of claim 60, wherein the additional anti-cancer therapy comprises chemotherapy.

62. The combination of claim 61, wherein the chemotherapy is fludarabine or cyclophosphamide.

63. The combination of claim 60, wherein the immunotherapy comprises an OX40 agonist, an IDO inhibitor, an anti-MERTK immunotherapy, an intratumoral injection, a STING-targeted immunotherapy, a NLRP3-targeted immunotherapy, a TLR9-targeted immunotherapy, a CPG-targeted immunotherapy, a TLR4-targeted immunotherapy, a TLR7/8-targeted immunotherapy, a OX40-targeted immunotherapy, a 4-1BB-targeted immunotherapy, a MER-TK-targeted immunotherapy, an oncolytic virus immunotherapy, an anti-CD40 immunotherapy, a FLT-3-ligand immunotherapy, or cytokine immunotherapies.

64. The combination of claim 63, wherein the cytokine immunotherapies are IL-2, IL-12, and/or IL-15.

65. The combination of any of claims 36-64, wherein the subject is a human.

66. The combination of any of claims 36-65, wherein the cancer is a lung cancer, a brain cancer, a breast cancer, a head and neck cancer, a cervical cancer, prostate cancer, a cancer of the eye, or a thyroid cancer.

67. The combination of claim 63, wherein the low dose radiation and/or immune checkpoint inhibitor are administered two or more times.
